(19) United States
(12) Patent Application Publication
(10) Pub. No.: US 2016/0184615 A1
(43) Pub. Date: Jun. 30, 2016

(54) Sonotrode

(71) Applicant: SÖRING GMBH, Quickborn (DE)

(72) Inventors: Florian Neumann, Hamburg (DE); Markus Meier, Elmshorn (DE); Ines Faber, Hamburg (DE)

(21) Appl. No.: 14/908,585

(22) PCT Filed: Jul. 21, 2014
(86) PCT No.: PCT/EP2014/065609

§ 371 Date: Jan. 29, 2016

(30) Foreign Application Priority Data
Jul. 30, 2013 (DE) 10 2013 214 944.1

(51) Int. Cl. A61N 7/00

(52) U.S. Cl. CPC A61N 7/00; A61B 2018/00577

(57) ABSTRACT

The invention relates to a sonotrode for an ultrasonic surgical instrument. The sonotrode comprises a shaft and an instrument head, which is located at a distal end of the shaft. A removal structure is arranged on the instrument head. A channel extends inside the shaft from an outlet opening on the instrument head to a channel opening. According to the invention, the channel opening is arranged in the circumferential surface of the sonotrode. Provision is additionally made that, in the area of the channel opening, an attachment part with a liquid connection is provided. By virtue of an attachment part being provided in the area of the channel opening, a rinse hose can be connected directly to the liquid connection of the attachment part, such that the hand part no longer comes into contact with the rinsing liquid. The cleaning work is thereby reduced.

### BACKGROUND

The invention relates to a sonotrode for an ultrasonic surgical instrument. The sonotrode comprises a shaft and an instrument head, on which a structure for ablating tissue is arranged. The instrument head is situated at a distal end of the shaft. A channel extends within the shaft from an outlet opening at the instrument head to a channel opening.

By way of example, such ultrasonic surgical instruments can be used for removing necrotic tissue and coatings. To this end, the sonotrode is connected to a handpiece. An ultrasonic transducer arranged in the handpiece generates high-frequency mechanical vibrations and transmits these to the connected sonotrode. As a result, the sonotrode is excited to vibrate. If the structure for ablating tissue arranged at the instrument head of the sonotrode is then brought into contact with the necrotic tissue, said tissue is ablated.

Undertaking a rinsing with a rinsing liquid during the procedure for the purposes of removing the ablated tissue is known. In the ultrasonic surgical instruments known from the prior art, a rinsing pipe guiding the rinsing liquid from the rinsing pipe into the channel of the sonotrode is arranged in the handpiece. The rinsing liquid then flows through the channel and emerges in the vicinity of the instrument head of the sonotrode in order to rinse ablated tissue away. Pressure variations produced by the ultrasound moreover lead to cavitation, which assists in the ablation of the tissue.

A disadvantage of this arrangement is that there needs to be sterile processing of not only the sonotrode but also the handpiece with the rinsing pipe arranged therein after the use of the ultrasonic surgical instrument.

### SUMMARY

The invention is based on the object of presenting a sonotrode which renders possible reduced cleaning outlay of the ultrasonic surgical instrument. Proceeding from the prior art set forth at the outset, the object is achieved by the features of the independent claims. Advantageous embodiments are found in the dependent claims.

According to the invention, the channel opening is arranged in the lateral surface of the sonotrode and an attachment piece is connected to the channel opening. The attachment piece is provided with a liquid connector. A liquid supplied to the liquid connector can enter into the channel of the sonotrode through the channel opening.

Using the attachment piece according to the invention renders it possible to connect a rinsing tube to the liquid connector of the attachment piece. A rinsing liquid guided through the rinsing tube can then flow into the channel opening of the sonotrode via the attachment piece and onward in the channel within the sonotrode. The rinsing liquid can emerge through the outlet opening arranged at the distal end of the sonotrode and it can remove ablated tissue. The invention has identified that a significantly more user-friendly and more economic use of the ultrasonic surgical instrument becomes possible by attaching an attachment piece in the region of the channel opening of the sonotrode. In the case of ultrasonic surgical instruments known from the prior art, it is necessary to guide the rinsing liquid through the handpiece. Since remains of the rinsing liquid remain in parts of the handpiece after use and since these liquid remains may form a nutrient medium for the multiplication of germs, it is necessary in the previously known ultrasonic surgical instruments for sterile processing of the handpiece to be carried out after each use. As a result of the attachment piece according to the invention, it is no longer necessary to guide the rinsing liquid through a rinsing pipe within the handpiece. Hence, the handpiece does not come into contact with the rinsing liquid or the patient and it is therefore not necessary for sterile processing to be carried out after use. This significantly reduces the outlay for sterile processing of the ultrasonic surgical instrument.

The sonotrode is configured to be connected to an ultrasonic transducer which makes the sonotrode vibrate. The connection between the sonotrode and the ultrasonic transducer is regularly established by way of the proximal end of the sonotrode, for example by virtue of the proximal end being inserted in a relevant receptacle. In order to enable this, the attachment piece is preferably at a distance from the proximal end of the sonotrode. The channel openings preferably have a distance from the proximal end of the sonotrode. Preferably, the channel openings and the attachment piece have substantially the same distance from the proximal end of the sonotrode. The distance is preferably selected in such a way that the channel openings are arranged in the vicinity of a vibration node of the sonotrode. A vibration node of the sonotrode denotes such a region of the sonotrode that is deflected as little as possible by the ultrasonic vibrations of the sonotrode. Channel openings arranged at this location lead to a small impairment of the vibration property of the sonotrode. Moreover, the connection stability between an attachment piece arranged in the vicinity of a vibration node and the sonotrode is impaired as little as possible by the ultrasonic vibrations. The distance between the channel openings and the sonotrode can be greater than 20%, preferably greater than 35% and more preferably approximately 50% of the length of the sonotrode.

The vibration property of the sonotrode depends on the design of the channel. It was found that it is advantageous for the vibration property of the sonotrode if the channel within the sonotrode is embodied substantially symmetrically relative to the vibration axis. The channel may comprise a main channel which, at least in sections, extends coaxially to the longitudinal axis of the shaft. Moreover, the channel may comprise a channel section which extends from the longitudinal axis of the sonotrode to the channel opening in the lateral surface. The channel portion can extend in a straight line. Preferably, provision is made for the channel section to include a right angle with the longitudinal axis of the shaft. Embodiments in which the channel section includes an angle with the longitudinal axis that is different from 90° are also comprised.

Preferably, a plurality of channel openings are provided in the lateral surface of the sonotrode. It is particularly advantageous for the channel openings to be arranged symmetrically around the longitudinal axis of the shaft. In this case, the term “symmetrical arrangement around the longitudinal axis” comprises an axial symmetry in relation to the longitudinal axis in the mathematical sense. However, furthermore, a rotational symmetry around the longitudinal axis about a specific angle is also comprised. The channel openings are then distributed uniformly around the longitudinal axis such that, after a rotation of the sonotrode around the longitudinal axis thereof about a specific angle, a channel opening respectively assumes the position which the adjacent channel opening had prior to the rotation.

Preferably, the channel sections which extend from the channel openings in the direction of the longitudinal axis of the shaft are arranged symmetrically in the aforementioned sense around the longitudinal axis of the sonotrode. By way of example, provision can be made for three channel openings to be provided, which are respectively offset by an angle of 120° in the circumferential direction. Disadvantageous effects on the vibration property of the sonotrode are avoided as a result of the symmetric arrangement of the channel openings and the channel sections leading to the channel openings. Preferably, the channel sections are formed by drilling.

During use, the ultrasonic surgical instrument is usually guided by a user and moved in any direction. Here, a secure connection between the attachment piece and the sonotrode is important. The attachment piece preferably encloses the sonotrode about the longitudinal axis thereof. Enclosing the sonotrode about the longitudinal axis thereof constitutes a simple type of connection, in which forces between the attachment piece and the sonotrode, which occur as a result of the movement of the sonotrode, can be absorbed in the radial direction. By way of example, the forces acting on the attachment piece in the longitudinal direction can be absorbed by way of frictional engagement with the sonotrode.

Preferably, provision is made for a sealing element which is arranged between the attachment piece and the sonotrode. A sealing element between the attachment piece and the sonotrode ensures a liquid-tight connection there. A liquid which enters the attachment piece from a tube by way of the liquid connector is therefore prevented from emerging in an unwanted manner. In order to prevent slippage of the sealing element relative to the sonotrode, the sealing element preferably engages in a groove situated in the lateral surface of the sonotrode. Preferably, two sealing elements are arranged between the attachment piece and the sonotrode such that the sealing elements enclose the channel opening therebetween. There can be frictional engagement between the attachment piece and the sealing element, by means of which the attachment piece is held on the sonotrode. Additional fastening means between the attachment piece and the sonotrode can then be dispensed with such that the attachment piece can easily be detached from the sonotrode and connected therewith. However, additional fastening means are not precluded.

The attachment piece and the two sealing elements preferably include a cavity therebetween, into which cavity the channel opening opens. The cavity can extend around the sonotrode. Preferably, the cavity extends as an annular space around the sonotrode. A liquid which reaches the attachment piece through the liquid connector can then spread in the annular space around the sonotrode. The liquid connector can be directed to the channel opening in order to enable a direct passage into the channel opening. However, provision can also be made for the liquid connector not to be directed to the channel opening. What is also ensured in this case is that a liquid which is guided into the attachment element is able to flow onward into the channel opening via the cavity.

Preferably, the liquid connector of the attachment piece is displaceable in the circumferential direction around the sonotrode. The liquid connector can then be brought into any angular position relative to the sonotrode. What is ensured in all angular positions is that the liquid can reach the channel opening since the annular space extends around the sonotrode in the circumferential direction and the channel opening opens into the annular space. The free adjustability of the angular position of the liquid connector and a liquid line connected thereto renders ideal operation of the ultrasonic surgical instrument possible for a user. The user can select the angular position in such a way that a connected tube interferes as little as possible during the procedure.

A mechanism, by means of which the alignment of the liquid connector can be set, can be provided within the attachment piece. In an advantageous embodiment, the attachment piece is designed in such a way that it can be rotated as a whole relative to the sonotrode. By way of example, this can be achieved by virtue of the frictional engagement between the attachment piece and the sonotrode being designed in such a way that it can be overcome by manual force. The sliding surface between the attachment piece and the sonotrode is preferably cylindrical.

In order to ease cleaning and sterilization of the sonotrode, the attachment piece is preferably connected to the sonotrode in a detachable manner. The attachment piece can then be detached from the sonotrode prior to cleaning and sterilization. The attachment piece can be a disposable article. Alternatively, the attachment piece is likewise sterilized and subsequently reconnected to the sonotrode.

Provision can be made for a liquid line, for example a tube, to be connectable to the liquid connector of the attachment piece with the aid of a plug-in connection. Alternatively, the liquid line can also be securely connected to the liquid connector of the attachment piece.

The sonotrode preferably consists of metal. The sonotrode can be configured as a turned part or, alternatively, as a milled part.

The subject matter of the invention moreover relates to an ultrasonic surgical instrument which comprises a sonotrode according to the invention and a handpiece. The handpiece is configured in a manner connectable to the sonotrode. The sonotrode is coupled to an ultrasonic transducer of the handpiece. When the attachment piece of the sonotrode is at a distance from the proximal end of the sonotrode, the proximal end can be inserted into the handpiece.

### BRIEF DESCRIPTION OF THE DRAWINGS

Below, a preferred embodiment of the invention is described in an exemplary manner with reference to the attached drawings. In detail:

FIG. 1 shows a lateral sectional view of an ultrasonic surgical instrument according to the invention.

FIG. 2 shows a sectional view in the transverse direction through the sonotrode of FIG. 1, wherein the section is taken in the region of the channel openings.

FIG. 3 shows a sectional view in the transverse direction through a further embodiment of the sonotrode, wherein the section is taken in the region of the channel openings.

### DETAILED DESCRIPTION

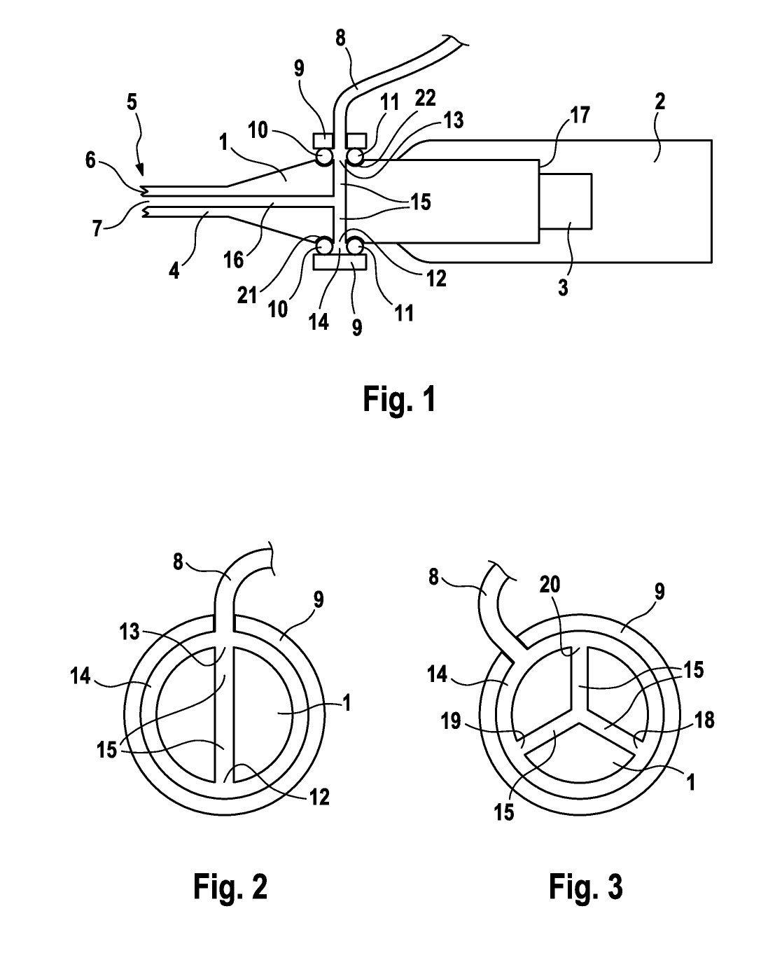

An ultrasonic surgical instrument in FIG. 1 comprises a handpiece 2, with the aid of which a user can guide the instrument. A sonotrode 1 is connected to the handpiece 2 by virtue of the proximal end 17 of the sonotrode 1 being screwed into the handpiece 2.

An ultrasonic transducer 3 is situated within the handpiece 2. The ultrasonic transducer 3 converts an electric AC voltage signal, obtained by a signal generator not depicted in FIG. 1, into a mechanical vibration. The vibration is transmitted to the sonotrode 1 and it propagates along the longitudinal direction via a shaft 4 to an instrument head 5 at the distal end of the sonotrode. An ablation structure 6 and an outlet opening 7 are arranged at the instrument head 5. If the vibrating ablation structure 6 is brought into contact with dead tissue, the tissue is ablated.

In order to remove the ablated tissue, a rinsing liquid can be supplied by way of a tube 8. The tube 8 is inserted into a liquid connector of an attachment piece 9. The attachment piece encloses the sonotrode 1 in a circular fashion. The attachment piece 9 is at a distance from the proximal end 17 of the sonotrode 1 so that the proximal part 17 of the sonotrode 1 can be used for establishing the screwed connection to the handpiece 2 without the attachment piece 9 being obstructive in the process.

Two sealing rings 10 and 11 are arranged between the sonotrode 1 and the attachment piece 9. The sealing rings 10, 11 are fastened to the sonotrode 1 by frictional engagement and they enclose the sonotrode 1 in a circular fashion. Here, the sealing rings 10, 11 respectively engage in a groove 21, 22. The grooves 21, 22 are let into the lateral surface of the sonotrode 1 in a circular fashion. Moreover, the attachment piece 9 is connected by way of frictional engagement with the sealing rings 10, 11. The sealing rings 10, 11 enclose an annular cavity 14 between them and the attachment piece 9. The attachment piece 9 and the annular cavity 14 are arranged in the region of channel openings 12, 13 which are situated in the lateral surface of the sonotrode 1. Hence, the channel openings 12, 13 open into the cavity 14. A channel section 15 respectively extends from the channel openings 12, 13 to a main channel 16. The two channel sections 15 meet the main channel 16 perpendicularly. The main channel 16 extends along the longitudinal axis of the sonotrode up to the outlet opening 7 arranged at the distal end.

A rinsing liquid supplied through the tube 8 can spread in the annular space 14 through the attachment piece 9. As a result of the connection between the annular space 14 and the channel sections 15 by way of the channel openings 12, 13, the rinsing liquid can then flow into the main channel 16. At the distal end, the rinsing liquid emerges by way of the outlet opening 7 and it can remove ablated tissue in the treated area by way of cavitation.

In the embodiment shown in FIG. 1, the liquid connector with the connected tube 8 is directed to the channel opening 13. However, it is also possible for the attachment piece 9, together with the liquid connector and the tube 8 connected thereto, to be rotated in the circumferential direction relative to the sealing rings 10, 11 or relative to the sonotrode. What is also ensured in other angular positions of the liquid connector is that the liquid reaches the outlet opening 7 via the annular space 14, the channel openings 12, 13 and onward by way of the channel sections 15 and the main channel 16 since the annular space 14 encloses the sonotrode 1 in a circular fashion.

FIG. 2 shows a cross section through the sonotrode 1 shown in FIG. 1, wherein the section was taken in the center of the channel openings 12, 13. The two channel openings 12, 13 are offset by an angle of 180° in the circumferential direction. The channel openings 12, 13 and the channel sections 15 are therefore arranged in a symmetrical manner in relation to the longitudinal axis of the sonotrode 1. What can also be seen in this illustration is that both the attachment piece 9 and the annular space 14 enclose the sonotrode 1 in a circular fashion. The liquid connector with the tube 8 plugged thereon is directed toward the channel opening 13. The sealing rings cannot be seen in this view since it shows a section which is situated precisely between the sealing rings.

FIG. 3 shows a cross section through a further embodiment of a sonotrode according to the invention. The section was likewise taken level with the channel openings. In this embodiment, three channel openings 18, 19, 20 are offset by an angle of 120° in the circumferential direction. When the sonotrode 1 is rotated through 120° about the longitudinal axis thereof, each channel opening assumes the space which an adjacent channel opening had prior to the rotation. Due to a relative twist of the attachment piece 9 in relation to the sonotrode 1, the liquid connector and the tube 8 plugged thereon are not directed to a channel opening, in contrast to the embodiment shown in FIG. 2. However, a rinsing liquid guided through the tube can nevertheless reach the channel sections 15 by way of the channel openings 18, 19, 20 due to the annular space 14. A user can freely select the angular position of the liquid connector and of the tube 8 such that the tube 8 interferes as little as possible during the procedure.

1-15. (canceled)

16. A sonotrode for an ultrasonic surgical instrument, comprising a shaft, an instrument head situated at a distal end of the shaft, an ablation structure arranged at the instrument head and a channel extending within the shaft from an outlet opening at the instrument head to a channel opening, wherein the channel opening is arranged in a lateral surface of the sonotrode and wherein an attachment piece with a liquid connector is connected to the channel opening, wherein the liquid connector is rotatable in a circumferential direction.

17. The sonotrode of claim 16, wherein a plurality of channel openings is formed in the lateral surface.

18. The sonotrode of claim 17, wherein the channel openings are arranged symmetrically in relation to a longitudinal axis of the shaft.

19. The sonotrode of claim 16, wherein the attachment piece surrounds the sonotrode.

20. The sonotrode of claim 16, wherein the attachment piece is at a distance from a proximal end of the sonotrode.

21. The sonotrode of claim 16, wherein the attachment piece is arranged in the region of a vibration node of the sonotrode.

22. The sonotrode of claim 16, wherein the attachment piece is connected by way of frictional engagement to the sonotrode.

23. The sonotrode of claim 16, wherein the attachment piece surrounds the sonotrode in a cylindrical manner.

24. The sonotrode of claim 16, wherein two sealing elements are arranged between the attachment piece and the sonotrode, which sealing elements enclose the channel opening therebetween.

25. The sonotrode of claim 24, wherein there is frictional engagement between the attachment piece and the sealing elements.

26. The sonotrode of claim 24, wherein the sealing elements and the attachment piece enclose a cavity therebetween, into which cavity the channel opening opens.

27. The sonotrode of claim 16, wherein the attachment piece is detachably connected to the sonotrode.

28. An ultrasonic surgical instrument, comprising a sonotrode for an ultrasonic surgical instrument, comprising a shaft, an instrument head situated at a distal end of the shaft, an ablation structure arranged at the instrument head and a channel extending within the shaft from an outlet opening at the instrument head to a channel opening, wherein the channel opening is arranged in a lateral surface of the sonotrode and wherein an attachment piece with a liquid connector is connected to the channel opening, wherein the liquid connector is rotatable in a circumferential direction, and a handpiece,
wherein the handpiece comprises an ultrasonic transducer and wherein the sonotrode is coupled to the ultrasonic transducer.

29. The ultrasonic surgical instrument of claim 28, wherein a proximal end of the sonotrode is inserted into a receptacle of the handpiece.

30. The sonotrode of claim 28, wherein a plurality of channel openings is formed in the lateral surface.

31. The sonotrode of claim 30, wherein the channel openings are arranged symmetrically in relation to a longitudinal axis of the shaft.

32. The sonotrode of claim 28, wherein the attachment piece surrounds the sonotrode.

33. The sonotrode of claim 28, wherein the attachment piece is at a distance from a proximal end of the sonotrode.

34. The sonotrode of claim 28, wherein the attachment piece is arranged in the region of a vibration node of the sonotrode.

35. The sonotrode of claim 28, wherein the attachment piece is connected by way of frictional engagement to the sonotrode.
